(19) United States
(12) Patent Application Publication
(10) Pub. No.: US 2017/0135863 A1
(43) Pub. Date: May 18, 2017

(54) Water Activated Cast Sock

(71) Applicants: Byeongsu Cheon, Ulsan (KR); Sangsoo Lee, Ulsan (KR)

(72) Inventors: Byeongsu Cheon, Ulsan (KR); Sangsoo Lee, Ulsan (KR)

(21) Appl. No.: 14/940,293

(22) Filed: Nov. 13, 2015

(51) Int. Cl. A61F 13/04; A61L 15/08; A61L 15/12; A61F 5/01; A61F 5/058

(52) U.S. Cl. CPC A61F 13/04; A61F 5/0109; A61F 5/0111; A61F 5/0118; A61F 5/05858; A61F 5/0585; A61L 15/12; A61L 15/08

(57) ABSTRACT

A breathable orthopedic cast having a variable width along its length is provided. The cast is formed of a breathable netting impregnated with a moisture activated hardener. Along the length the cast has a reduced width portion to aid in conforming over a joint such as an ankle.

### BACKGROUND OF THE INVENTION

Field of the Invention

The present invention relates generally to medical casts. More particularly the present invention relates to a water activated cast sock having tapered regions to fit to body contours.

Description of Related Art

Traditional casts typically depend upon skilled personnel, such as physicians, manually building a cast to protect and align a part of the body of a human or other animal containing a fractured bone, thereby facilitating proper fusion of the fractured bone. A common method for building casts involves applying a sheep skin cloth, stockinette, or equivalent (collectively, “stockinette”) in the area around the fractured bone (“desired area”), and iterations of impregnating swaths of cloth, usually cotton, with a hardening agent, such as hardening latex or a synthetic resin (collectively, “hardening agent”), and of layering the swaths of cloth, before the swaths harden, around the stockinette. The hardening of the hardening agent (originally Plaster of Paris, but today synthetic resins are more commonly used) permeating the layered swaths transforms the layered swaths into a cast. After each application of cloth swathing hardens, another layer is applied in similar fashion until the desired cast stiffness is obtained.

The traditional method of building a cast has several problems: First, personnel skilled in cast-making are required, especially in creating the edges of a cast, spacing the spirals of swathing, judging the number of layers necessary for protection of the bone fusion process, and abrading the surface of each successive layer to eliminate gaps between layers. This is a time-consuming, difficult process. Second, traditional casts do not “breathe” very well, i.e., the hardened cast does not allow the free exchange of air between the skin surface under the cast and the atmosphere. Poor cast breathing can lead to itchy or even infected skin conditions. Third, casts with irregular surfaces can cause blistering, and worse, improper fusion of bones. Fourth, traditional casts are radio-opaque, that is, X-rays cannot penetrate a traditional cast, which prevents X-ray imaging through the cast. The inability to use X-ray imaging deprives the treating physician of information about healing of bone fractures. Fifth, traditional casts can not be subjected to prolonged contact with water, so bathing or showering by the cast-wearer becomes problematic.

Further, casting often encounters difficulties due to the contours of human or animal body parts, such as tapering around ankles, knees, wrists, elbows and the like. As such, there has long been a need for a simpler method of cast building, especially one that did not require highly skilled personnel, created comfortable cast edges, permitted breathing, and minimized irregularities in cast construction.

Therefore, what is needed is a device that may easily be formed as a breathable cast capable of quickly and easily contouring to the shape of a body part being supported by the cast.

### SUMMARY OF THE INVENTION

The subject matter of this application may involve, in some cases, interrelated products, alternative solutions to a particular problem, and/or a plurality of different uses of a single system or article.

In one aspect, a breathable cast assembly positioned on a limb and a joint is provided. The limb is an elongate limb such as an arm or leg, and has a joint with a width that is less than the limb before and after the joint. For example a wrist or an ankle. A cast is positioned over the limb and the joint. The cast comprises a tubular sleeve having two open ends. The sleeve is formed of a netting fabric, which is impregnated with a water-activated material. The tubular cast sleeve further defines at least one inward tapering region along its length. The inward tapering region forms an area of the tubular sleeve with a reduced diameter or width (depending on if it has a circular or other cross section) from the remainder of the sleeve. The inward tapering region is followed along the length by an outward tapering region which tapers to an increased diameter or width from the reduced diameter or width. The tubular sleeve thereby defines an hourglass shape, which is positioned over the joint of the limb.

In another aspect, a breathable cast sleeve is provided. The cast sleeve is formed of a tubular sleeve which has two open ends that is formed of a netting fabric. The netting fabric is impregnated with a water-activated material that will harden when exposed to water and dried. Further, the tubular sleeve defines at least one inward tapering region along its length. The inward tapering region forms an area of the tubular sleeve with a reduced diameter or width (depending on if it has a circular or other cross section) from the remainder of the sleeve. The inward tapering region is followed along the length by an outward tapering region which tapers to an increased diameter or width from the reduced diameter or width. The tubular sleeve thereby defines an hourglass shape.

### BRIEF DESCRIPTION OF THE DRAWINGS

FIG. 1 provides a view of and embodiment of the present invention.

FIG. 2 provides an elevation view of another embodiment of the present invention.

FIG. 3 provides a perspective view of an embodiment of the present invention on a limb of a person.

FIG. 4 provides a detail view of an embodiment of a netting used for the present invention.

### DETAILED DESCRIPTION

The detailed description set forth below in connection with the appended drawings is intended as a description of presently preferred embodiments of the invention and does not represent the only forms in which the present invention may be constructed and/or utilized. The description sets forth the functions and the sequence of steps for constructing and operating the invention in connection with the illustrated embodiments.

Generally, the present invention concerns a tubular cast sleeve formed of a diamond or netting pattered fabric. This fabric is impregnated with a moisture activated polyurethane resin which can be activated by water, and will harden when dried, thus forming a breathable and rigid cast. Particularly, the present invention has at least one tapered region having the tubular shape decrease in diameter along the length of the sleeve. The tapered portion then increases in diameter, forming an hourglass shape. This tapered portion is configured to easily adapt to reduced diameter portions of a human or animal body, such as an ankle, wrist, knee, elbow, and the like. This structure may be used for patients with orthopedic fractures, diabetic ulcers, and the like. The breathability of the present invention allows for faster healing time and a more comfortable healing experience for the wearer. Thus this variable width cast sleeve makes application and reduces application time.

In a particular embodiment, the tubular sleeve is formed of a diamond pattern netting of polypropylene or polyolefin that is blended with a stretchable fabric. This netting fabric is impregnated with the moisture-activated polyurethane resin. An tapered region of the sleeve forms an hourglass shape with an inward and then outward tapering to make the tubular sleeve easier to conform to a body part, such as a leg, for example an ankle area. Also the threads may be water resistant, thereby limiting bacterial and other microbial growth. Further, the tubular sleeve may have multiple layers of the flexible layers, and in one embodiment, may have between 4 and 9 layers.

In some embodiments, a separate, flexible and breathable skin protector may be applied below the cast sleeve. This breathable skin protector may be any fabric or material capable of allowing air to pass through it. The skin protector, like the tubular sleeve, may also have a tapered region, such that its diameter decreases and then increases along the length of the sleeve, forming an hourglass shape.

The tapering portion may be positioned anywhere along the length of the tubular sleeve. In one embodiment, the tapering portion may be at approximately ½ of the length of the sleeve. In another embodiment, the tapering portion may be at approximately ⅓ of the length of the sleeve.

In an embodiment of operation, the present invention may be soaked in water briefly, for example for 5-15 seconds. The sleeve may be slid or, in some embodiments rolled on. Rolling embodiments typically require that the sleeve be pre-rolled (such that they have a donut shape), and then placed in position and rolled up the limb until fully unrolled. The soaking in water may be performed either before or after the rolling. In some embodiments, the tubular sleeve of the cast may be provided in a pre-rolled configuration.

In other embodiments, the tubular sleeve cast may be applied dry, and then may be wetted in place.

Once applied, in one embodiment a skin protector which was previously applied to the limb before application of the cast can be folded over the cast sleeve (the skin protector having a length greater than that of the sleeve). Next, the cast may be molded into tight fitting position about the limb for approximately 3 minutes until it begins to set. In some embodiments, water may continue to be added during the fitting process.

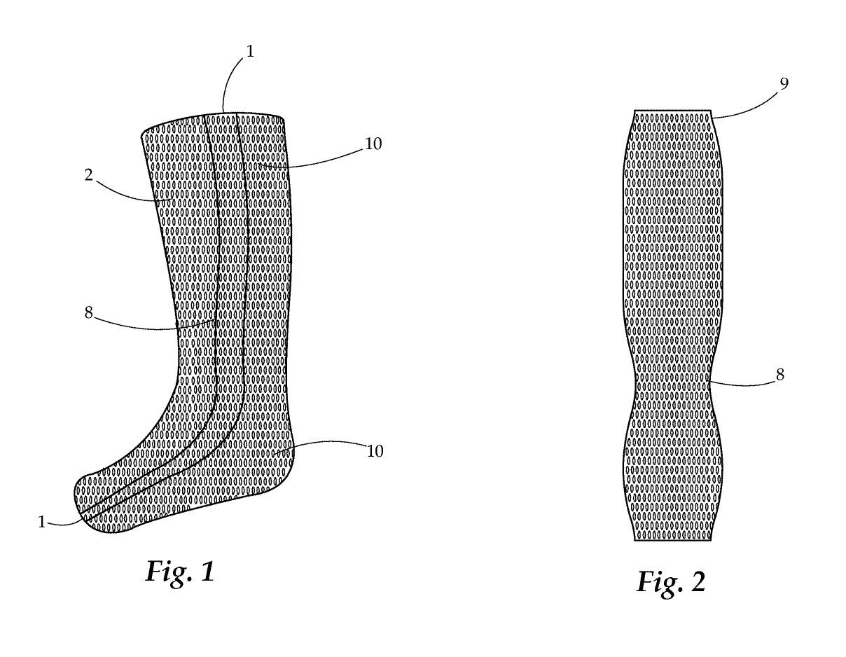

Turning now to FIG. 1, a view of the cast of the present invention is shown in the shape of a foot and lower leg cast. In this view it can be seen that there is a tapered portion 8, and portions having greater diameters 10. The cast sleeve 1 is formed of a diamond mesh 2, which is impregnated with a water-activated material that will harden when exposed to water and dried, such as a resin material.

FIG. 2 provides an elevation view of an embodiment of the tubular sleeve 1. In this view, the sleeve is flat and has not been wetted or shaped. The tapered region 8 forms an hourglass shape between the wider regions 10. Further, in this embodiment the sleeve also tapers inward at its end 9. It should be understood that there may be more than one tapering region and in some cases the taper may not return to a wider diameter area, but may maintain the tapering shape to the end, or may straighten along the length of the sleeve.

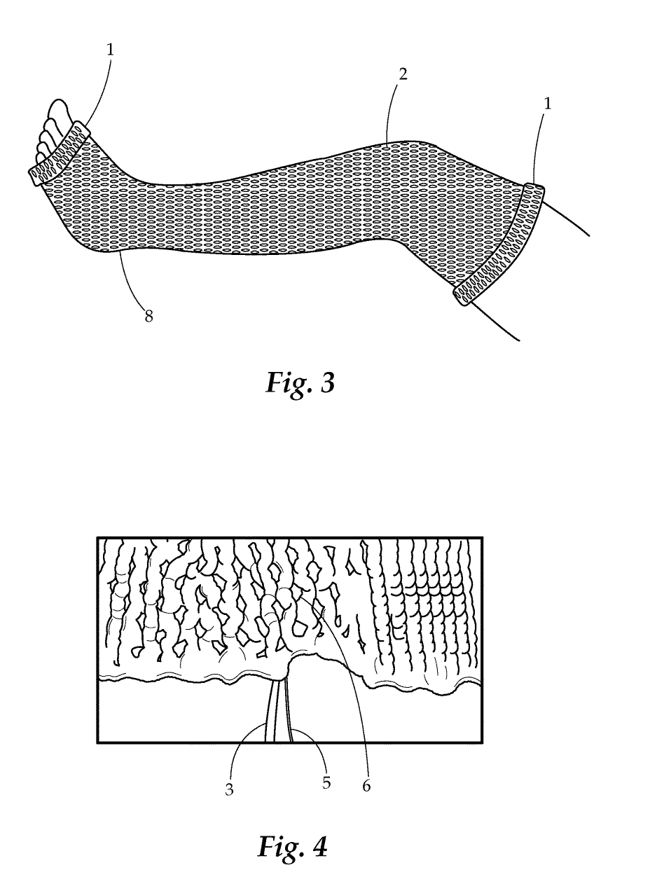

FIG. 3 provides a view of the present invention applied to a leg and ankle of a patient. The diamond mesh 2 sleeve 1 has been rolled from a rolled position up or down the limb to be fully elongated and applied to the leg. Narrowed region 8 is positioned over the ankle of the wearer. Narrowed region 8 allows the sleeve 1 to comfortably fit and be easily fitted to the leg of the patient.

In some cases, as can be seen in FIG. 3, sleeve 1 can be rolled over itself at the ends to size and reinforce the ends of the cast when formed. Further, the skin protector (not shown) can be longer than the sleeve and can be folded over the sleeve 1 when forming the final cast.

FIG. 4 provides a detail view of the netting fabric of the sleeve 1. The diamond shape 6 of the netting is shown, as well as the ends of the netting 3. Water-activated impregnation 5 is impregnated into the netting.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

What is claimed is:

1. A breathable cast sleeve comprising:
a tubular sleeve having two open ends, the sleeve being formed of a netting fabric, the netting fabric being impregnated with a water-activated material that will harden when exposed to water and dried;
wherein the tubular sleeve defines at least one inward tapering region along a length, the inward tapering region forming an area of the tubular sleeve with a reduced diameter from the remainder of the sleeve, the inward tapering region being followed along the length by an outward tapering region which tapers to an increased diameter from the reduced diameter, the tubular sleeve thereby defining an hourglass shape.

2. The breathable cast sleeve of claim 1 wherein the netting fabric is a diamond netting.

3. The breathable cast sleeve of claim 1 wherein the water-activated material is a polyurethane resin.

4. The breathable cast sleeve of claim 1 wherein the tubular sleeve further comprises an inwardly tapering region at one of the two ends of the sleeve.

5. The breathable cast sleeve of claim 1 wherein the tubular sleeve further comprises an inwardly tapering region at both ends of the sleeve.

6. The breathable cast sleeve of claim 1 wherein the at least one inwardly tapering region is positioned at ⅓ down a length of the sleeve.

7. The breathable cast sleeve of claim 1 wherein the at least one inwardly tapering region is positioned at ½ down a length of the sleeve.

8. The breathable cast sleeve of claim 1 further comprising a skin protector, the skin protector being a thin breathable tubular fabric, the skin protector positioned on an inside of the tubular sleeve.

9. The breathable cast sleeve of claim 8 wherein the skin protector has a length that is greater than the tubular sleeve, and wherein the skin protector protrudes from the two open ends of the sleeve.

10. The breathable cast sleeve of claim 1 wherein the netting fabric comprises one of a polypropylene and a polyolefin mixed with a stretchable fabric.

11. A breathable cast assembly positioned on a limb and a joint comprising:
a limb, the limb being elongate and having a joint; and
a cast, the cast positioned over both the limb and the joint, the cast comprising:
a tubular sleeve having two open ends, the sleeve being formed of a netting fabric, the netting fabric being impregnated with a hardened water-activated material;
wherein the tubular sleeve defines at least one inward tapering region along a length, the inward tapering region forming an area of the tubular sleeve with a reduced diameter from the remainder of the sleeve, the inward tapering region being followed along the length by an outward tapering region which tapers to an increased diameter from the reduced diameter, the tubular sleeve thereby defining an hourglass shape, the inward tapering region positioned directly over the joint.

12. The breathable cast assembly of claim 11 wherein the netting fabric is a diamond netting.

13. The breathable cast assembly of claim 11 wherein the water-activated material is a polyurethane resin.

14. The breathable cast assembly of claim 11 wherein the tubular sleeve further comprises an inwardly tapering region at one of the two ends of the sleeve.

15. The breathable cast assembly of claim 11 wherein the tubular sleeve further comprises an inwardly tapering region at both ends of the sleeve.

16. The breathable cast assembly of claim 11 wherein the at least one inwardly tapering region is positioned at ⅓ down a length of the sleeve.

17. The breathable cast assembly of claim 11 wherein the at least one inwardly tapering region is positioned at ½ down a length of the sleeve.

18. The breathable cast assembly of claim 11 further comprising a skin protector, the skin protector being a thin breathable tubular fabric, the skin protector positioned on an inside of the tubular sleeve.

19. The breathable cast assembly of claim 18 wherein the skin protector has a length that is greater than the tubular sleeve, and wherein the skin protector protrudes from the two open ends of the sleeve.

20. The breathable cast assembly of claim 11 wherein the netting fabric comprises one of a polypropylene and a polyolefin mixed with a stretchable fabric.
